(19) United States
(12) Patent Application Publication
(10) Pub. No.: US 2012/0029302 A1
(43) Pub. Date: Feb. 2, 2012

(54) APPARATUS AND METHOD FOR DYNAMIC DETECTION OF ELECTROPHYSIOLOGICAL SIGNALS FOR STILLNESS FEEDBACK

(76) Inventors: Shih-Chung Hsu, Tainan City (TW); Shu-Min Hsu Yang, Tainan City (TW)

(21) Appl. No.: 12/846,276

(22) Filed: Jul. 29, 2010

(51) Int. Cl. A61B 5/04

(52) U.S. Cl. 600/300

(57) ABSTRACT

A training device aims to indicate a targeted object to be gazed at by a trainee, then trainee's visual sight to the targeted object is obstructed. After a selected duration a hint is given to the trainee to execute command actions related to the targeted object. Through completeness degree of trainee's execution of the command actions trainee's stillness can be evaluated and trained. During measuring electrophysiological signals such as brainwaves, trainee's feedback command actions can serve as a basis for measurement of electrophysiological signals.

### FIELD OF THE INVENTION

The present invention relates to an apparatus and method for dynamic detection of electrophysiological signals for stillness feedback to train stillness and serve as basis of electrophysiological signal measurements.

### BACKGROUND OF THE INVENTION

Researches indicate that emotional and psychological obstacles of many contemporary people are caused by concentration and memory problems. Concentration and memory affect learning effectiveness, daily life independence, emotional control and interpersonal relationship in groups, and also affect sleep quality and health.

In recent years, advance of technology makes measurement of electrophysiological signals in a non-invasive manner possible to get physiological signals, such as breath, heart beats and pulse, brainwaves and the like to get more understandings of people's physical conditions.

Take brainwaves for instance, they are generally categorized according the frequency in α, β, θ and δ waves, or the measurement values are integrated and processed to become attention waves and meditation waves of brainwave signals. In practice, the brainwaves have been used in psychological and educational fields to measure attention. One method being adopted is to send stimulation signals such as pictures, sound and movies to a trainee, and measure trainee's brainwave alterations upon receiving the stimulation.

However, measurement of brainwaves can only detect brain activity degrees of the trainee, but cannot detect spiritual variations of the trainee under the same degree of brain activities. Hence many therapies or trainings based on the brainwave measurement can only serve as reference data in analysis, therapy and training processes, but cannot be used as decisive guideposts.

In short, the method of using the conventional electrophysiological signal measurement apparatus such as measuring brainwaves or the like has a big flaw in practice, i.e. it can only get relationship of sending the stimulation signals and the electrophysiological signal activities of the trainee such as brainwaves, but cannot detect trainee's received conditions. When the stimulation signals are sent during the analysis, therapy and training process, the effectiveness of the stimulation signals could be impaired due to many factors, such as trainee's personal preference, mood or desire. And erroneous interpretation and judgment of the actual conditions could occur.

### SUMMARY OF THE INVENTION

The primary object of the present invention is to provide training for capability to concentrating on a selected object (i.e. stillness) and a basis of measurement to overcome the restriction on measuring electrophysiological signal at present.

To achieve the foregoing object the present invention adopts a technique as follow: based on research findings, a person can achieve an optimal condition for learning in a completed concentration moment. Moreover, with attention focusing on a single object memory power also improves, thus can indirectly improve the efficiency of handling a task, sentiment, lift attitude and the like, and also can improve sleep quality, and enhance inherent self-healing and immunity. Hence stillness training is a vital issue in the life and learning of people in the modern society.

Therefore the present invention provides a training device for stillness that includes a response zone on an upper surface to indicate at least one targeted object and a waiting zone surrounding the response zone. When a trainee takes training or test, the trainee first gazes at the targeted object and places a specific limb (such as fingers) on the waiting zone; after a first hint is given, trainee's visual sight to the targeted object is obstructed; then a second hint is given; the trainee moves the specific limb in the condition of obstructing visual sight to execute command actions related to the targeted object (such as pointing the fingers to the targeted object); the response zone of the training device detects completion degree of the command actions and evaluates a trainee's stillness value. Through multiple times of training and testing trainee's mental stability and attention can be adjusted to improve the stillness.

In another aspect, the present invention can be incorporated with an electrophysiological signal detection device such as a brainwave test device to receive electrophysiological signals of the trainee and send the electrophysiological signals to the training device.

As the conventional electrophysiological signal (such as brainwaves) detection or training method can only passively receive stimulation, and cannot detect trainee's mood during training and test, trainee could feel irritated, bored or lack of desire and low cooperation that cause decreasing of accuracy of measurement results, and results in discrepancy and irrational phenomena of outward symptoms (small or big difference) vs. brainwave measurement values (big or small variation).

To solve the aforesaid problems, the present invention requests the trainee to provide active feedback by executing the specific command actions, and then measure the electrophysiological signals to enhance the accuracy of the measured value. Moreover, during training and test period the electrophysiological signal detection device continuously measures the electrophysiological signals to adjust and train the trainee to be inured in a physically and spiritually relaxed condition without other unrelated thoughts to get desired attention naturally, namely “natural attention”.

The present invention, in one embodiment, can be complemented with the brainwave test device to continuously measure brainwave values resulted from discharge of trainee's brain during training and test processes, or the brainwave values can be integrated and calculated to get brainwave signals such as attention waves and meditation waves. When the trainee executes the command actions related to the targeted object through the training device, incorporating with the brainwave measurement results, trainee's brainwave alterations during executing the command actions can be compared and obtained. Through the stillness training and brainwave measurement whether trainee's stillness in attention is achieved through arduous effort or natural attention got in a relaxed condition can be known. By observing the brainwave alterations in repeated training and adjustment for a long duration trainee can be trained to achieve natural attention and stillness desired.

As most people's attention focuses on a certain matter, the important issue of training is concentration. In practice, human's body and spirit can get optimal learning effect and self-healing power in the natural condition. When people is irritating in mind or physically fatigue, although it is still possible to concentrate by effort, it is under unnatural pressure and stress, even if concentration can be achieved under such a stressed condition, the physical and spiritual conditions cannot fully take the advantage of optimal learning and training opportunity. Not only learning effect suffers, a negative thought could be created or seeds of illness could be sowed.

Moreover, when the trainee is unwilling to receive training and test, or is distracted and cannot concentrate, paying attention to execute the command actions of the training device is difficult. Through evaluation of the training device trainee's stillness feedback value can be known not reaching a selected standard, and the brainwave measurement result thereof does not have accuracy or reference value.

In another aspect, if the trainee tries to influence training and measurement results to control brainwaves by consciousness, although the measurement result of brainwaves could be impacted, incorporating with the result of executing the command actions through the training device, the artificial change of the brainwave measurement result can be excluded.

The present invention makes the brainwave measurement on a credible common basis, namely measures trainee's brainwave values by performing active feedback and executing same command actions, hence can sift the brainwave measurement result with improved credibility, then arrange training procedures according to trainee's physical and mental conditions.

In short, the present invention can achieve many benefits, notably:

1. The present invention provides a standard to measure natural attention and stillness to clearly indicate trainee's natural attention and stillness.

2. The present invention provides a measurement basis for electrophysiological signal detection devices, and generates actual electrophysiological signals with research and reference value or provides a high credibility dynamic measurement method on such a basis, and provides valuable breakthrough in illness prevention and therapy and analysis.

3. The present invention can train trainee's natural attention and adjusts trainee's concentration habit to help the trainee to be inured to concentrate and relax in a natural relaxed condition.

4. The present invention trains trainee's stillness and helps the trainee to organize thought and concentrate.

5. The present invention restores trainee's natural attention and stillness, and eliminates negative thought, and restores self-confidence, loving mind and sleep quality, and indirectly restores self-healing and immunity power.

The foregoing, as well as additional objects, features and advantages of the present invention will be more readily apparent from the following detailed description, which proceeds with reference to the accompanying drawings.

### BRIEF DESCRIPTION OF THE DRAWINGS

FIG. 1 is a schematic view of the present invention in an operating condition.

FIG. 2 is a flowchart of process steps of the present invention.

FIG. 3 is a schematic view of an embodiment of the present invention in an operating condition.

FIG. 4 is a flowchart of an embodiment of the process steps of the present invention.

### DETAILED DESCRIPTION OF THE PREFERRED EMBODIMENT

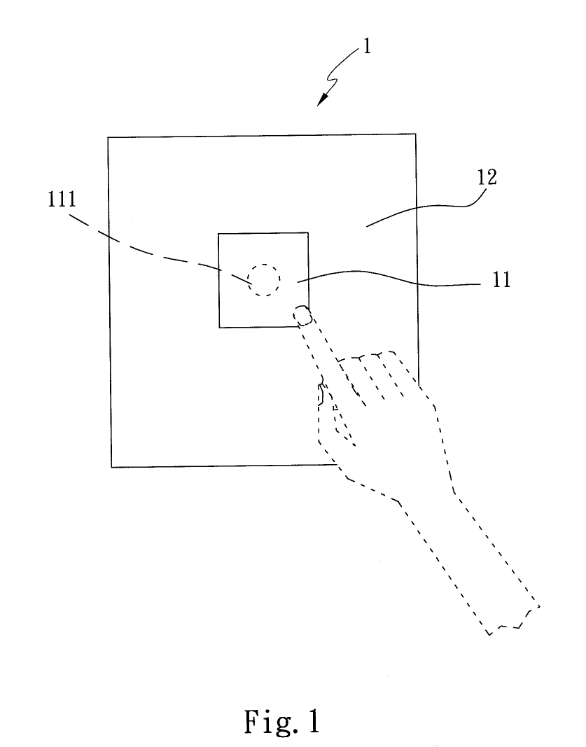

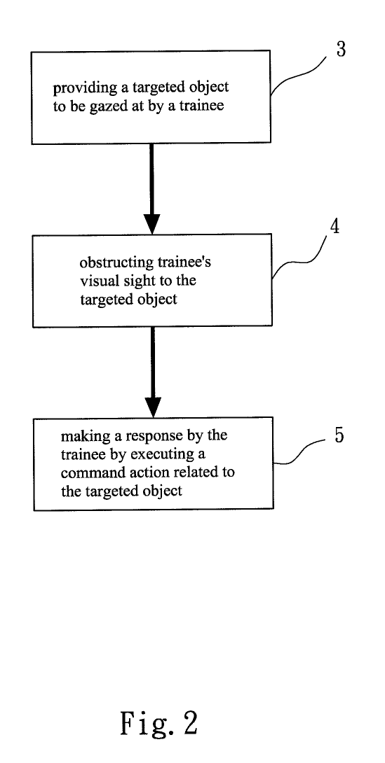

Referring to FIGS. 1 and 2, an apparatus for dynamic detection of electrophysiological signals for stillness feedback of the present invention includes a training device 1 with a response zone 11 located on an upper surface thereof and a touch device or touch screen located beneath the response zone 11 to hold a picture of at least one targeted object 111. The targeted object 111 may be more than one. The response zone 11 is surrounded by a waiting zone 12.

A method for dynamic detection of electrophysiological signals for stillness feedback according to the present invention includes steps as follow:

step 1 (3): provide the targeted object 111 to be gazed at by a trainee, the trainee points fingers to the waiting zone 12 of the training device 1;

step 2 (4): provide a first hint and obstruct the visual sight of the trainee to the targeted object 111; after the trainee has received the first hint and the visual sight of the trainee to the targeted object is obstructed, the trainee continuously pays attention to the location of the targeted object 111; the first hint can be an audition hint (sound or voice), a visual hint or a tactile hint; and

step 3 (5): provide a second hint and the trainee makes a response, and execute command actions related to the targeted object; the executed command actions could be moving the fingers to point out the location of the targeted object 111 in the response zone 11; the second hint can be an audition hint (sound or voice), a visual hint or a tactile hint.

Alternatively, the trainee could use a stylus to point out the location of the targeted object 111 in the response zone 11.

After the trainee has executed the aforesaid steps a number of times, calculate distance difference of trainee's recognition and the actual targeted object 111 to get trainee's stillness. By repeatedly use the training device 1 and method of the present invention, a dynamic test and training of trainee's stillness can be performed. Through the training the trainee is inured rapidly in a non-distracted state to enhance stillness.

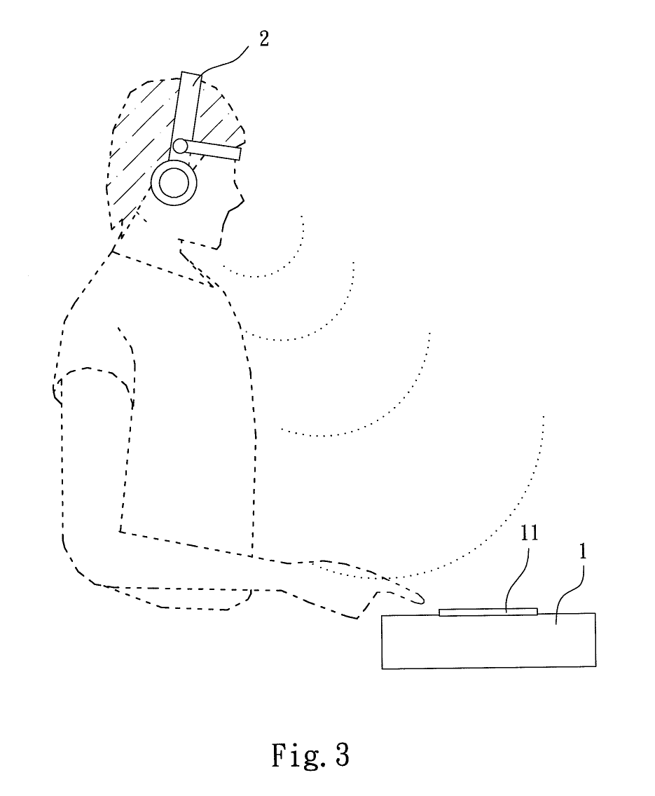

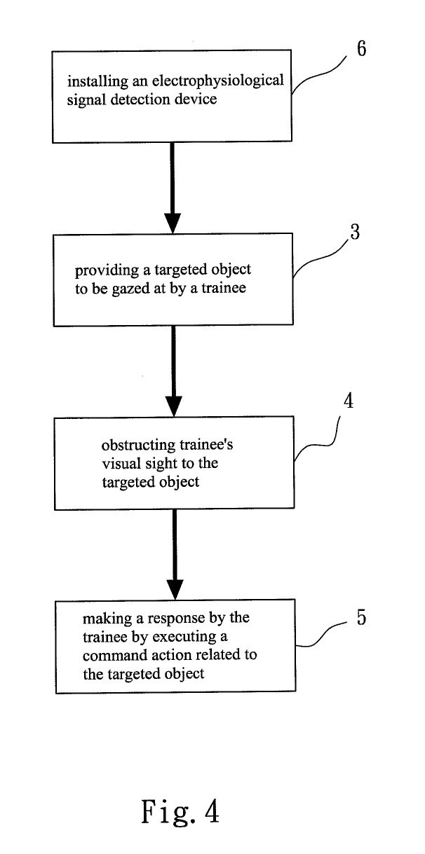

Refer to FIGS. 3 and 4 for an embodiment of the present invention. It employs an electrophysiological signal detection device 2 such as a brainwave test device to continuously measure alterations of a trainee's electrophysiological signal such as brainwaves during the training process. The signal received by the electrophysiological signal detection device 2 is sent to the training device 1.

The aforesaid embodiment is implemented according the steps as follow:

step 1 (6): install the electrophysiological signal detection device 2 to continuously measure dynamic alterations of the electrophysiological signals of the trainee during the training process;

step 2 (3): provide the targeted object 111 to be gazed at by the trainee, and the trainee points fingers to the waiting zone 12 of the training device 1;

step 3 (4): after the first hint is given, obstruct trainee's visual sight to the targeted object 111; after the trainee has received the first hint and the visual sight to the targeted object 111 is obstructed, the trainee continuously pays attention to the location of the targeted object 111; the first hint can be an audition hint (sound or voice), a visual hint or a tactile hint; and

step 4 (5): provide the second hint and the trainee executes the command actions related to the targeted object 111; the second hint can be an audition hint (sound or voice), a visual hint or a tactile hint, and the executed command actions could be moving the fingers to point out the location of the targeted object 111 in the response zone 11.

In the aforesaid embodiment the electrophysiological signal detection device 2 such a brainwave test device continuously receives trainee's electrophysiological signals such as brainwaves that are compared with the dynamic electrophysiological signals (such as attention wave signals and meditation wave signals) of the trainee during executing the aforesaid steps 1 through 4 on the training device 1. The training device 1 integrates evaluated stillness results based on the command actions to judge electrophysiological signal alterations of the trainee upon reaching a certain degree of stillness. The method of the present invention, through the obtained feedback value or feedback value basis of the same degree, can dynamically measure the actual or meaningful electrophysiological signals of the trainee.

In the general condition, when the trainee is executing step 1, gazing at the targeted object 111 will release attention wave signal from trainee's brain; during executing step 2, after trainee's visual sight is obstructed, the brainwave releases meditation wave signals; at step 3, the trainee moves the fingers to execute the command actions related to the targeted object 111 while visual contact with the targeted object 111 is obstructed, stillness value can be measured and attention wave signals and meditation wave signals can be compared to detect trainee's actual brain electrophysiological signals.

When the trainee has been trained to a set stillness degree, the trainee has concentration capability. The stillness value in such a condition can serve as a basis. When the trainee has reached a certain stillness value standard, whether trainee's stillness is natural attention can be determined through the attention wave signals and meditation wave signals of the trainee. Trainee's physical and mental conditions such as mood and habits can also be adjusted to gradually train trainee's natural attention.

In the event that the trainee has no desire to go through the training, and his/her attention has been directed to other matters, attention wave signals are intensified. Stillness value will notably indicate impact of such distractions. If the trainee tries to influence training and measurement results by purposefully control brainwaves through will power, although the attention wave signals and meditation wave signal will be affected, the stillness value will indicate distraction effect caused by purposeful control of the brainwaves.

What is claimed is:

1. An apparatus for dynamic detection of electrophysiological signals for stillness feedback, comprising:
a training device which includes a response zone on an upper surface to indicate at least one targeted object and a waiting zone surrounding the response zone; and
an electrophysiological signal detection device which received electrophysiological signals of a trainee and sent the electrophysiological signals to the training device.

2. The apparatus for dynamic detection of the electrophysiological signals for stillness feedback according to claim 1, wherein the training device further includes pictures of the targeted object beneath the response zone.

3. The apparatus for dynamic detection of the electrophysiological signals for stillness feedback according to claim 1, wherein the response zone is a touch screen.

4. A method for dynamic detection of electrophysiological signals for stillness feedback, comprising the steps of:
step 1: providing a targeted object to be gazed at by a trainee;
step 2: obstructing trainee's visual sight to the targeted object; and
step 3: making a response by the trainee by executing a command action related to the targeted object.

5. The method for dynamic detection of the electrophysiological signals for stillness feedback according to claim 4, wherein the step 1 is preceded by another step of installing an electrophysiological signal detection device.

6. The method for dynamic detection of the electrophysiological signals for stillness feedback according to claim 4, wherein a first hint is provided before obstructing trainee's visual sight to the targeted object.

7. The method for dynamic detection of the electrophysiological signals for stillness feedback according to claim 6, wherein the first hint is selected from the group consisting of an audition hint, a visual hint and a tactile hint.

8. The method for dynamic detection of the electrophysiological signals for stillness feedback according to claim 4, wherein a second hint is provided before making a response by the trainee.

9. The method for dynamic detection of the electrophysiological signals for stillness feedback according to claim 8, wherein the second hint is selected from the group consisting of an audition hint, a visual hint and a tactile hint.

10. The method for dynamic detection of the electrophysiological signals for stillness feedback according to claim 4, wherein executing the command action is moving trainee's limbs to point out the location of the targeted object in the response zone.

11. The method for dynamic detection of the electrophysiological signals for stillness feedback according to claim 10, wherein the limb is trainee's fingers.

12. The method for dynamic detection of the electrophysiological signals for stillness feedback according to claim 4, wherein executing the command action is using a stylus to point out the location of the targeted object in the response zone.
